(19) United States
(12) Patent Application Publication
(10) Pub. No.: US 2013/0345499 A1
(43) Pub. Date: Dec. 26, 2013

(54) SIDE TO SIDE ANASTOMOSIS

(71) Applicants: Cornelis Antonius Franciscus Tulleken, Utrecht (NL); Rutger Joan Bart Tulleken, Bussum (NL)

(72) Inventors: Cornelis Antonius Franciscus Tulleken, Utrecht (NL); Rutger Joan Bart Tulleken, Bussum (NL)

(73) Assignee: AMJ B.V., Utrecht (NL)

(21) Appl. No.: 13/978,115

(22) PCT Filed: Jan. 12, 2011
(86) PCT No.: PCT/NL2011/050019

§ 371 Date: Sep. 16, 2013

(51) Int. Cl. A61F 2/06

(52) U.S. Cl. CPC A61F 2/064; A61F 2/062
USPC 600/36

(57) ABSTRACT

The invention relates to an assembly comprising on the one hand a graft vessel and on the other hand an anastomosis has connector. The connector comprises a ring and two pins. Each pin has a free end and a fixed end connected to the ring. The pins extend, viewed from the fixed end towards the free end, next to and set apart from each other. Each pin has an overlap section where the pin, viewed in axial direction of the ring, overlaps the plane bounded by the outer contour of the ring. The axial axis of the ring extends transverse to the wall of the graft vessel. The connector is attached to the graft vessel and the ring lies, with its full circumference, against the wall of the graft vessel. The invention further relates to a method of providing such an assembly and a method of attaching such an assembly to a recipient vessel.

The invention relates to the field of side-to-side (abbreviated as STS) anastomosing.

A STS-anastomosis is a in the medical field a connection between two channels, in general blood vessels, which are connected with their sides against each other. In the region where the sides of the vessels lie against each other there is an aperture allowing blood to flow from the one vessel into the other vessel. STS-anastomoses are frequently used in the field of bypass surgery for example on the heart.

According to the older medical prior art these kind of anastomoses with a graft vessel and recipient vessel are made as follows: in each vessel an opening is made in the vessel wall; these openings are laid onto each other with the openings overlapping each other; and subsequently the graft and recipient vessels are attached to each other by suturing. This is a time consuming operation and in order to prevent spillage of blood the recipient vessel need to be shut off from blood flow—like by clipping—. According to more recent medical prior art connector devices have been developed to simplify making the STS-anastomosis. Up to now, these connector devices do not seem to be used widely spread. Apparently, these connector devices still do not fulfil the needs and requirements of safety and reliability.

The object of the invention is to provide a reliable manner for making an anastomosis. According to a first aspect, this object is achieved by providing an assembly of a graft vessel and an anastomosis connector according to claim 1. According to a second aspect, this object is achieved by providing a method of making (such) an assembly according to claim 22. And according to a third aspect, this object is achieved by providing a method of making a STS-anastomosis according to claim 32.

All these aspects of the invention have in common, that use is made of an “anastomosis connector of the type comprising a ring and two pins”; wherein each pin has a free end and a fixed end connected to the ring, wherein the pins extend, viewed from the fixed end towards the free end, next to and set apart from each other; and wherein each pin has an overlap section where the pin, viewed in axial direction of the ring, overlaps the plane bounded by the outer contour of the ring.

An “anastomosis connector of the type comprising a ring and two pins” is as such known from WO 2009/123434. Also the not yet published PCT/NL2010/050778 describes an anastomosis connector of this type. Both WO 2009/123434 and PCT/NL2010/050778 are fully incorporated in this application for detailed description of embodiments of the anastomosis connector as can be used with the present invention. With respect to these earlier PCT applications, it is noted that the applicant AMJ B.V. of this application is also the applicant of WO 2009/123434 and PCT/NL2010/050778.

Like in WO 2009/123434 and/or PCT/NL2010/050778, also in the “anastomosis connector of the type comprising a ring and two pins” as used in the present invention:
at least part of said overlap sections can extend parallel to the ring, in a first position of the pins with respect to the ring; and/or
the pins can be rigidly connected to the ring; and/or
the pins can extend in the same direction;
the pins, can with respect to the ring, be moveable from a first position to a second position in order to form between on the one hand the ring and on the other hand the pins an opened jaw; and in which the pins are moveable from the second position to the first position towards the ring in order to close the jaw; and/or
in the second position, the overlap sections of the pins can be at an angle with respect to the plane defined by the ring in order to form a sad jaw which, viewed from the fixed end towards the free end, widens, and wherein the pins are swivelable, with respect to the ring, from the second position to the first position; and/or
the anastomosis connector can further comprises a tensioning member which, when the pins are in the second position, pretensions the pins towards the first position; and/or
the free ends of the pins can be are arranged at a distance from the overlap section, which distance is at least 50% of the radius of the ring, in particular at least 100% of the radius of the ring; and/or
the sections of the pins between the overlap section and the free end of each pin, can extend essentially parallel to each other.

It is however, to be noted that not only the above summarized embodiments, but all embodiments described and/or shown in shown WO 2009/123434 and PCT/NL2010/050778 can also be used with the present invention.

An assembly according to claim 1 enables making a side-to-side anastomosis connection in an easy manner which requires relative little time and results in a very safe and reliable anastomosis. For making the anastomosis, one just has to pierce the free ends of the pins from the outside of the recipient vessel through the wall of the recipient vessel into the lumen of the recipient vessel and subsequently sliding the assembly further up until the pins fully passed through the wall of the recipient vessel. In case a tensioning member is present, like according to PCT/NL2010/050778, this will be followed by releasing the tensioning member for closing the jaw formed between the pins and ring. For piercing the free ends of the pins, one can insert these free ends through bores already present in the wall of the recipient vessel. More practical however is to make the bores for inserting the free ends into the recipient vessel with the free ends itself, which free ends for this purpose are optionally pointed and/or sharp so that the free ends easily penetrate through the wall of the recipient vessel. Controlling the location where the free ends pierce through the wall of the recipient vessel with accuracy is easy. Easy piercing and/or controlling the accuracy can be increased by gripping a part of the wall of the recipient vessel, at the location where the anastomosis is to be made, with a gripper—like a pair of pincers, a pair of tweezers or any other suitable medical instrument—and lifting or pulling the gripped part a little. Although it is conceivable that first an opening in the wall of the recipient vessel is made in order to allow—after attaching the assembly—flow connection between the lumen of the recipient vessel and graft vessel, the assembly according to the invention allows to make this opening for said flow connection after attaching the assembly according to the invention to the wall of the recipient vessel. This allows testing the connection for example on tightness and being leak free. In case the connection might not meet the testing criteria, the assembly according to the invention can be removed from the recipient vessel or left unused as it is on the recipient vessel without proceeding further. In case the assembly according to the invention might be removed from the recipient vessel—without the opening for flow connection through the wall of the recipient vessel being made—leak through the pierced bores will be minimal if present at all. In case of such leak, this will be easy to stop if necessary or otherwise it will heal from itself. Further, the anastomosis connector of the type used according to the invention ensures a tight and leak free connection as is already explained in the earlier mentioned WO 2009/123434 and PCT/NL2010/050778 of AMJ B.V.

After having attached the graft vessel to the side wall of the recipient vessel, the flow connection between the lumen of the graft vessel and the lumen of the recipient vessel can be made in case not already made in advance of attaching the graft vessel to the recipient vessel. In order to make the flow connection, the part of the wall of the recipient vessel [—which part is in this application also named the flap part—], which part is located in the region which is—viewed in axial direction of the ring—so to say surrounded by the ring, is to be provided with an opening or to be removed. Said flap part can easily be accessed via one of the ends of the graft vessel and the lumen of the graft vessel. One simply inserts a cutting instrument into this end of the graft vessel and passes this cutting instrument up to the flap part to be removed or provided with an opening. The cutting instrument can be provided with a sharp knife or other sharp cutting tool. The cutting instrument can however also be a laser, like an Excimer laser, which can burn an opening through the wall of the recipient vessel. For example reference can be made to the laser as disclosed in EP-750476 and U.S. Pat. No. 5,964,750, which is very suitable for use with the present invention. During making an opening in the flap part or removing the flap part, the ring of the connector can serve on the one hand as an abutment preventing the cutting instrument to enter the recipient vessel to far and/or on the other hand for keeping the flap part flat and/or tensioned. Keeping the flap part flat and/or tensioned during cutting assists in more easy and reliable making of the opening in the flap part or removal of the flap part.

Using a laser, like an Excimer laser, as cutting tool for cutting an opening in the wall of the recipient vessel and/or in the wall of the graft vessel results in a smooth transition from graft lumen to recipient lumen with very limited or no exposure of cellular structures of the vessel wall (like collagen, elastine, muscle cells, etc) because due to the laser energy these structures are kind of sealed off. Due to this effect rapid overgrowth of neo-intima will take place.

After finishing the anastomosis connection, one end of the graft vessel can be closed by clip or other suitable means. In general this will be the end which was used for accessing the flap part. At the other end the graft vessel will in general be connected to another vessel. This connection of the other end to another vessel can be present already before the anastomosis connection according to the invention is made, but it can also be made afterwards. Further, at the other end of the graft vessel the connection with a further vessel can optionally also be a side-to-side anastomosis according to this invention.

With respect to the graft vessel it is to be noted that according to the invention this can be an artificial vessel or a biological vessel. According to the invention, the graft vessel can also be made from a combination of biological and artificial material. In case of a biological vessel, it is noted that this can originate from a human or animal donor—other than the patient itself—, but it can also originate from the patient itself, like a saphenous vein from the leg or an internal mammary artery from the chest. The graft vessel can according to the invention also be a vessel having a biologically cultured cell layer grown onto a supporting tube, like a harness or gauze structure.

Further it is noted that the assembly according to the invention, also in case a graft vessel originating from the patient itself, can be prepared completely outside the patient even without being connected in any manner to the patient. The assembly according to the invention can of course be made in the operation room during the operation just beside he patient itself, but it can also be made remote from the patient in a laboratory, factory or other suitable facility during or before the operation.

According to a further embodiment of the assembly according to the invention, the wall of the graft vessel is provided with an opening surrounded by the ring. This means that the flap part of the graft vessel is already removed before the assembly according to the invention is mounted to the recipient vessel. As already stipulated above, according to the invention this opening can have been formed by laser cutting an opening into the wall of the graft vessel.

According to a further embodiment of the assembly according to the invention, the connector is attached to the graft vessel by sutures attaching the ring onto the wall of the graft vessel.

According to a further embodiment of the assembly according to the invention, the connector is attached to the graft vessel by adhesive adhering the ring onto the wall of the graft vessel. This attachment by adhesive can according to the invention also be combined with the above mentioned attachment by sutures but the attachment with adhesive can also be without attachment by sutures.

According to a further embodiment of the assembly according to the invention, the connector is attached to the graft vessel by the pins piercing through the wall of the graft vessel. This attachment by the pins can according to the invention also be combined with the above mentioned attachment by sutures and/or adhesive, but the attachment with the pins can also be without attachment by sutures and/or adhesive. Using the pins for attachment of the connector to the graft vessel allows, after attachment of the assembly according to the invention to the recipient vessel, wall tissue of both the recipient vessel and the graft vessel being clamped between the pins and the ring of the connector. This improves the tightness of the connection as well the connection being leak free. Further this supports—after the operation—growth of the tissue of the graft vessel and recipient vessel into integral new tissue.

According to a further embodiment of the assembly according to the invention, the pins extend in the length direction of the graft vessel. This allows the pins to be longer than the diameter of the ring. Pins longer than the diameter of he ring are practical when attaching the assembly according to the invention to the recipient vessel.

According to a further embodiment of the assembly according to the invention, the ring lies against the outer surface of the wall of the graft vessel. This is easy to establish. Further it allows the operator when attaching the assembly to the recipient vessel a good view on the location of attachment.

According to a further embodiment of the assembly according to the invention, the a part of the wall of the graft vessel lying inside the region surrounded by the ring covers the inside of the ring. The pins might stay uncovered. It might even be folded over 180° around the ring to fully cover the ring whilst leaving the pins uncovered. In this way, internal wall tissue of the graft vessel will prevent blood, flowing through the anastomotic connection, from contacting the ring.

According to a further embodiment of the assembly according to the invention, the ring lies against the inner surface of the wall of the graft vessel and the pins lie outside the graft vessel and extend parallel to the outer surface of the wall of the graft vessel. As indicated above, this allows, after attachment of the assembly according to the invention to the recipient vessel, wall tissue of both the recipient vessel and the graft vessel being clamped between the pins and the ring of the connector. This improves the tightness of the connection as well the connection being leak free. Further this supports—after he operation —growth of the tissue of the graft vessel and recipient vessel into integral new tissue.

According to a further embodiment of the assembly according to the invention, the graft vessel has a first end and a second end; wherein the pins, viewed from the fixed ends towards the free ends of the pins, extend towards the second end; and wherein the distance from the connector to the first end of the graft vessel is smaller than the distance from the connector to the second end. This allows when, an applicator is inserted through the same end of the graft vessel as through which the connector has been inserted, good manipulation of the assembly according to the invention as one has a good view onto the pins. Insertion of the applicator and the connector through the same end of the graft vessel, prevents the inner tissue of the graft vessel at the other side of the connector being damaged. In order to allow sufficient vessel length for closing the first end of the graft vessel after establishing the anastomosis connection, the distance from the connector to the first end is at least 0.5 cm. When a catheter—like an Excimer laser catheter as is for example disclosed in EP-750476 and U.S. Pat. No. 5,964,750—is used for making the fluid connection between the lumens of the graft vessel and recipient vessel, the distance from the connector to the first end is at least 1.0 cm.

According to a further embodiment of the assembly according to the invention, a rod like application member could be inserted through an end of the graft vessel into the lumen of the graft vessel.

According to the second aspect, the invention provides a method of making an assembly comprising on the one hand a graft vessel and on the other hand an anastomosis connector; wherein the connector comprises a ring and two pins; each pin having a free end and a fixed end connected to the ring; the pins extending, viewed from the fixed end towards the free end, next to and set apart from each other; each pin having an overlap section where the pin, viewed in axial direction of the ring, overlaps the plane bounded by the outer contour of the ring;

wherein the method comprises the step of:
arranging the ring to lie, with its full circumference against the wall of the graft vessel and with its axial axis transverse to the wall of the graft vessel; and
attaching the connector to the graft vessel.
   With this method an assembly according to the invention can be made.

According to a further embodiment of the method of the second aspect of the invention, further comprising the step of making an opening through the wall of the graft vessel in the region surrounded by the ring. As already indicated above, the step of making said opening can comprise burning with a laser, such as an Excimer laser, an opening through the wall of the graft vessel.

According to a further embodiment of the method of the second aspect of the invention, the step of attaching the connector to the graft vessel comprises:
suturing the ring onto the wall of the graft vessel; and/or
adhering the ring onto the wall of the graft vessel; and/or
piercing the pins through the wall of the graft vessel.

According to a further embodiment of the method of the second aspect of the invention, the step of attaching the connector to the graft vessel comprises:
inserting the connector into the lumen of the graft vessel with the free ends of the pins pointing in the direction of insertion;
piercing the free ends of the pins from the inside of the graft vessel through the wall of the graft vessel to outside the graft vessel and subsequently shifting the connector further in the direction of insertion up to the pins fully passed through the wall of the graft vessel and the ring lies against the inner surface of the wall of the graft vessel.

According to a further embodiment of the method of the second aspect of the invention the ring is placed against the outside of the graft vessel when performing the step of attaching the connector to the graft vessel. In this embodiment, the step of attaching the connector to the graft vessel can comprise the step of covering the inside of the ring with a part of the wall of the graft vessel lying inside the region surrounded by the ring.

According to the third aspect, the invention provides a method of making a side-to-side anastomosis, comprising the step of:
attaching an assembly according to the first aspect of the invention to a recipient vessel by piercing the free ends of the pins from the outside of the recipient vessel through the wall of the recipient vessel into the lumen of the recipient vessel and subsequently sliding the assembly further up until the pins fully passed through the wall of the recipient vessel; and
subsequently making a flow connection between the lumen of the graft vessel and the lumen of the recipient vessel.

According to a further embodiment of the method according to the third aspect of the invention, the step of making said flow connection comprises burning with a laser, such as an Excimer laser, an opening through the wall of the recipient vessel and/or graft vessel.

According to a further embodiment of the method according to the third aspect of the invention, the laser comprises a tubular bundle of densely packed laser fibres arranged to burn a ring and a suction gripper arranged inside the tubular bundle for gripping the part bounded by the ring burnt with the laser, and wherein the step of burning comprises burning a ring shaped passage through the wall of the recipient vessel and/or graft vessel and suctioning the part bounded by the ring burnt into the tubular bundle. The ‘part bounded by the ring burnt into the tubular bundle’ is the earlier mentioned flap part. Such a laser with a tubular bundle of densely packed laser fibres and capable of gripping the flap part by suctioning is for example disclosed in EP-750476 and U.S. Pat. No. 5,964,750, which are incorporated by reference for further details of an example of such a laser.

According to a further embodiment of the method according to the third aspect of the invention, the assembly is, during the step of attaching, being manipulated by means of an an application member. This application member can for example be a rod like application member inserted through one end of the graft vessel into the lumen of the graft. The rod like application member can for example be a catheter, like a laser catheter as disclosed in EP-750476 and U.S. Pat. No. 5,964,750. Another example of such an application member is a surgical gripping tool, like a tweezers, forceps or needle holder. Such a gripping tool can grip the ring of the connector. In case the ring of the connector lies inside the graft vessel, the gripping tool might be inserted into the graft vessel to grip the ring directly or the gripping tool might grip the ring from the outside together with tissue of the graft vessel.

According to a further embodiment of the method according to the third aspect of the invention, the insertion direction of the application member is the same as the direction into which the pins of the connector extend. This allows good manipulation of the assembly of graft and connector as one has a good view onto the pins.

According to a further embodiment of the method according to the third aspect of the invention, the rod extends in the direction of the axial axis of the ring and the part of the graft vessel extending from adjacent the connector in the direction of the other end of the graft vessel is kept parallel to the application member. This allows a good view onto the location where the anastomosis is to be made so that the operator can see what he is doing.

According to a further embodiment of the method according to the third aspect of the invention, a part of the wall of the recipient vessel is, at the location where the anastomosis is to be made, gripped and lifted or pulled, transverse to the longitudinal axis of the recipient vessel, away from said longitudinal axis. This makes the piercing easier and/or allows improved control of the accuracy. Although in some cases this gripping can be done with the fingers of the hand of the operator, in general a gripper—like a pair of pincers, a pair of tweezers or any other suitable medical instrument—will be used.

According to a further embodiment of the method according to the third aspect of the invention, the parts of the graft vessel adjacent the connector are folded towards the axial axis of the ring, during the step of attaching. These parts might even be kept alongside each other. As explained above in relation to the rod like application member, this enables a good view onto the location where the anastomosis is to be made. Said parts of the graft vessel adjacent the connector can however also be kept alongside each other without a rod like applicator being inserted into one of the ends of the graft vessel.

According to a further embodiment of the method according to the third aspect of the invention, the method further comprises a step of providing a said assembly according to the first aspect of the invention. According to still a further embodiment, this step of providing said assembly comprises the method according to the second aspect of the invention.

The invention will now be further elucidated with reference to the drawings. In these drawings:

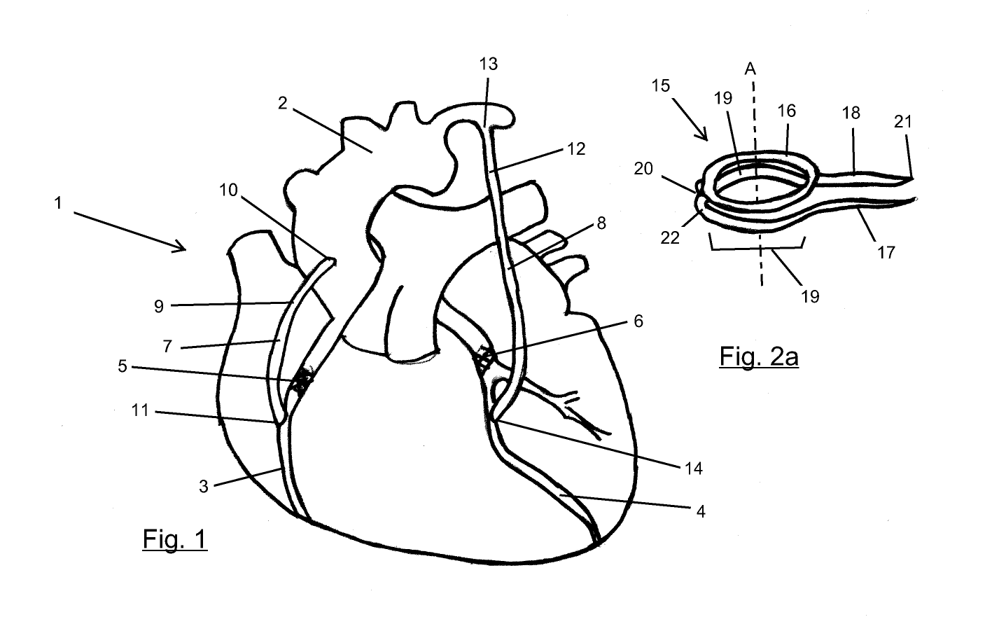

FIG. 1 shows a schematic view of a human heart with two bypasses;

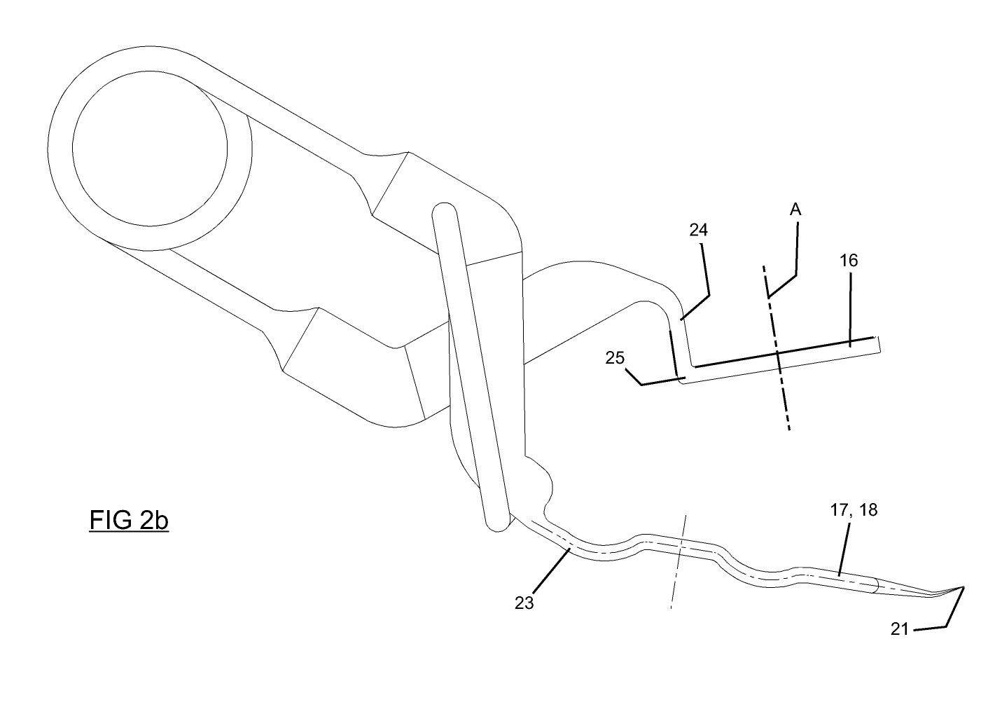

FIG. 2 shows a schematic perspective view of two connector as can be used with the invention; FIG. 2a shows one example and FIG. 2b shows another example;

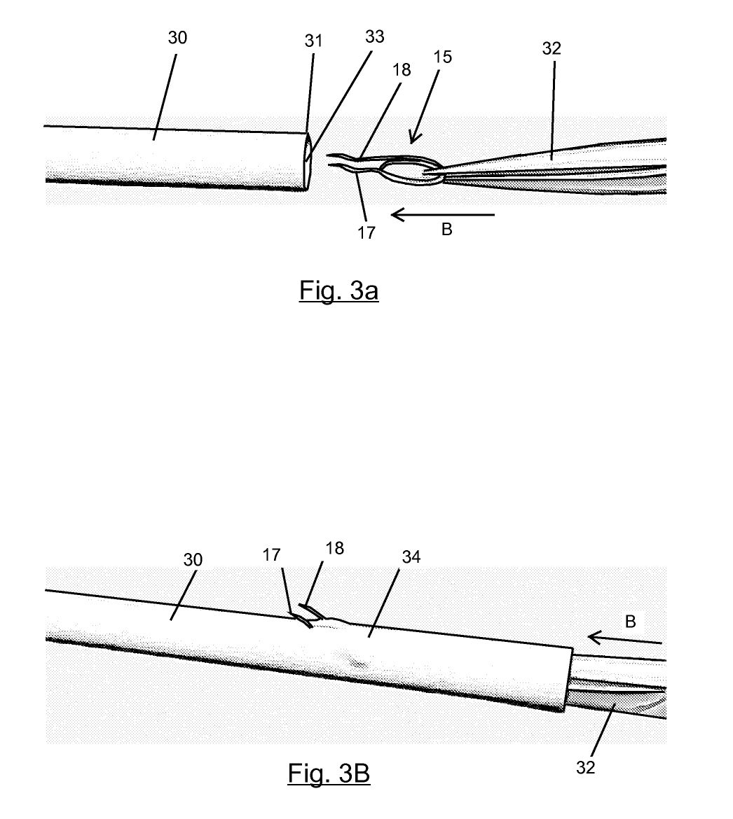

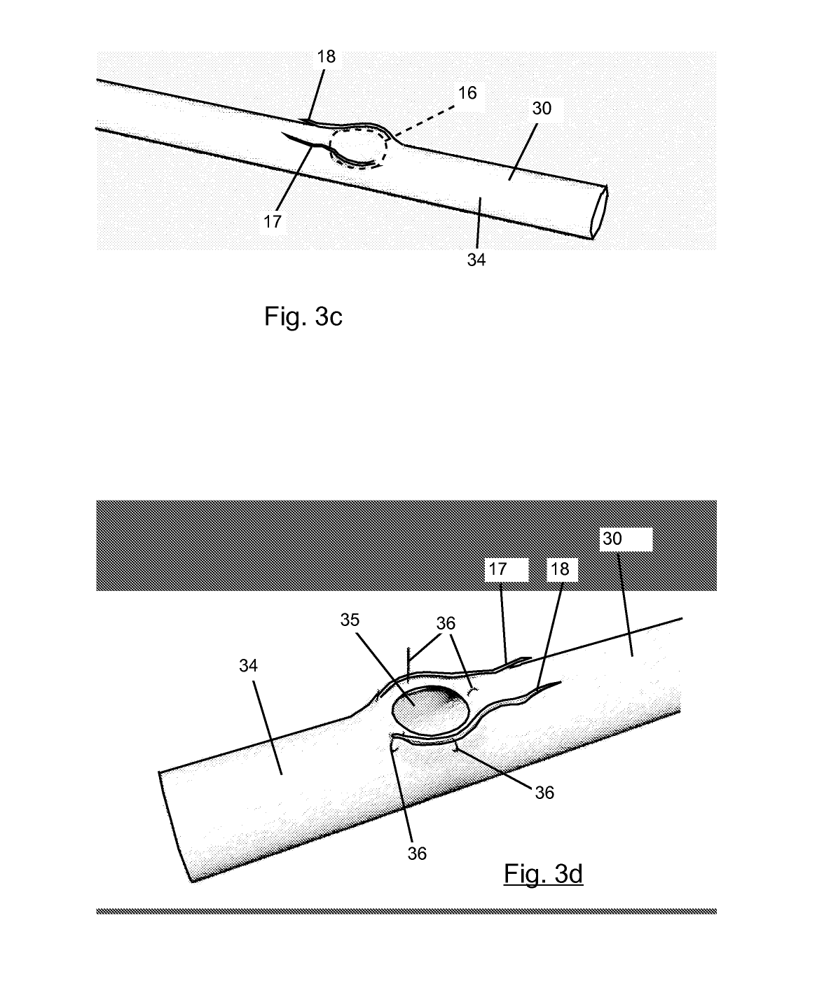

FIG. 3 illustrates with schematic perspective views a first method of making an assembly according to the invention, the FIGS. 3a, 3b, 3c and 3d each illustrate subsequent steps;

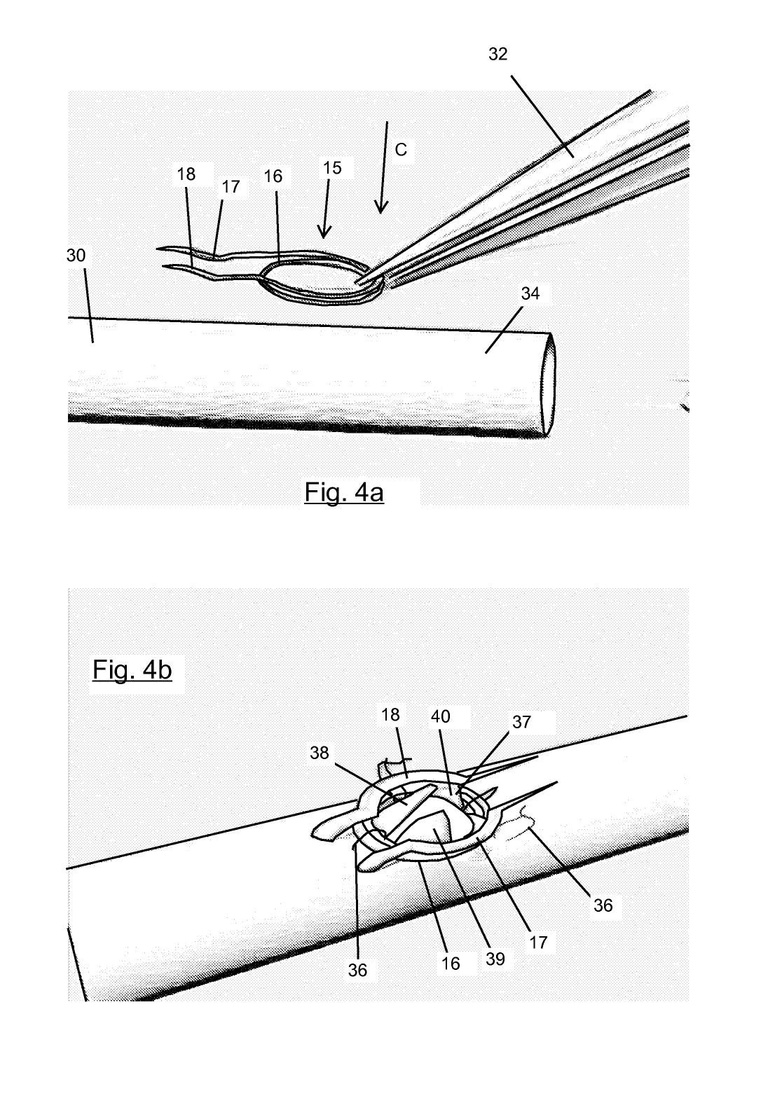

FIG. 4 illustrates with schematic perspective views a second method of making an assembly according to the invention, the FIGS. 4a and 4b each illustrate subsequent steps; and

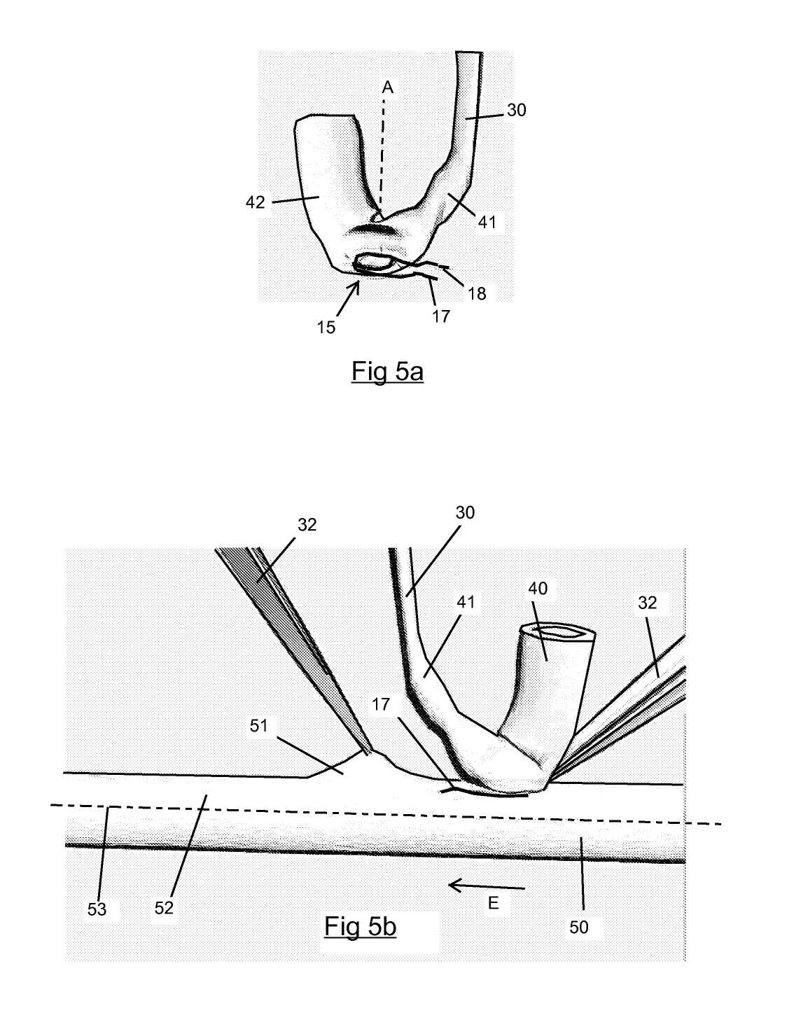

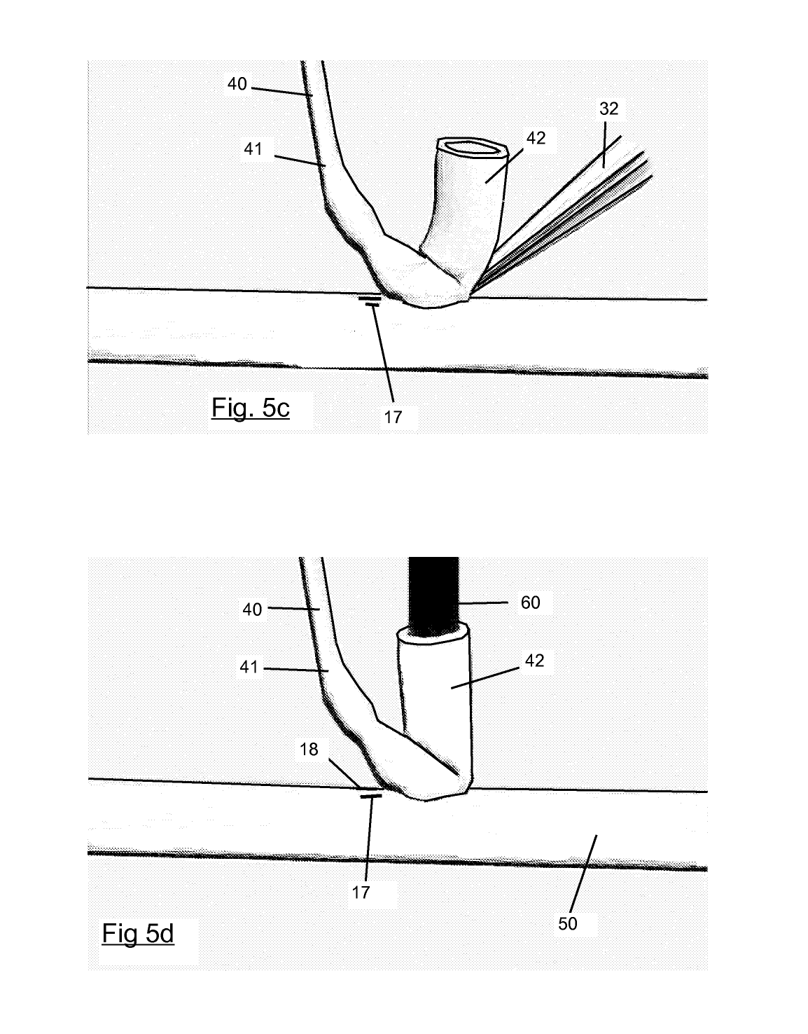

FIG. 5 illustrates with schematic perspective views a method of making a side to side anastomosis according to the invention, the FIGS. 5a, 5b, 5c and 5b each illustrate subsequent steps.

FIG. 1 shows schematically a human heart 1. No. 2 indicates the aorta and no. 3 and no. 4 are both a coronary artery which is blocked at 5 respectively 6. In order to restore circulation through the blocked coronary arteries 3 and 4, a bypass 7 has been made to bypass blockage 5 and a bypass 8 has been made to bypass blockage 6.

For bypass 7 a saphenous vein from the leg has been used as graft vessel 9. The anastomotic connections at 10 and 11 are made according to the present invention and will be described further below. Graft vessel 9 can be prepared according to the invention completely outside and separated from the body of the patient. This preparation can for example take place at a location where the patient is not present, but it can also take place close to the patient in the operating room.

The term ‘the graft vessel being separated from a human or animal body’ and the term ‘the graft vessel being separated from the body of the patient’ as used in this application means that the graft vessel is a separate entity not attached to a human/animal/patient body. Of coarse these term(s) do not exclude that somebody can keep it in his hand or manipulate it. These term(s) mean that the graft is, when separated, not part of the biological system of a body of a human/animal/patient.

For bypass 8 an internal mammary artery from the chest has been used as graft vessel 12. At location 13 this graft vessel has a natural connection to the aorta. Thus at this location no artificial anastomotic connection is required. At location 14 an anastomotic connection according to the present invention has been made. Graft vessel 12 can according to the invention be prepared inside the body of the patient or completely outside the body of the patient, but graft vessel 12 will stay connected at location 13 with the patient.

In FIG. 1 the coronary artery 4 is the recipient vessel with respect to graft vessel 12 and coronary artery 3 and aorta 2 are each a recipient vessels with respect to graft vessel 9.

FIG. 2a shows schematically a connector 15 as can be used according to the invention. This connector 15 comprises a ring 16 and two pins 17, 18 which are with the so called fixed end 22 attached to the ring 16 at 20, and extend from the fixed end 20 towards the pointed free end 21, next to and set apart from each other in the same direction. The pins 17, 18 have an overlap section 19 in which they overlap the plane bounded by the ring. The plane bounded by the ring is as such defined as the plane ‘surface’ lying inside the ring and the ring itself. The plane bounded by the ring thus does not extend outside the ring. Axis A is the axial axis of the ring.

A connector 15 like the one as shown in FIG. 2a is also known from WO 2009/123434, see for example the FIGS. 5 and 6 of WO 2009/123434. In fact all the securing devices as disclosed in WO 2009/12434 can be used as a connector for the present invention. The complete document WO 2009/12434 is therefore incorporated by reference for further details of connectors according to the invention.

Although the connector 15 shown in FIG. 2a is also shown in the FIGS. 3, 4 and 5—to be discussed below, it is noted that connectors of the according to the invention can also be of different design, as follows from the example of the connector shown in FIG. 2b. FIG. 2b is identical to FIG. 6a of PCT/NL2010/050778. For details of FIG. 2b reference is made to PCT/NL2010/050778. It is noted that in fact all devices for making an anastomosis connection as described, claimed and shown in PCT/NL2010/050778 can be used as a connector for the present invention. The complete document WO 2009/12434 is therefore incorporated by reference for further details of connectors according to the invention.

Referring to FIG. 2b of the present application, it might be considered to extend 23 and to arrange at 25 between the ring 16 and leg 24 a similar extension about parallel to 23 in order to create more distance between on the one hand the ring 16 and pins 17, 18 and on the other hand the leg 24.

FIG. 3 illustrates a first method of making an assembly according to the invention.

FIG. 3a shows a graft vessel 30, the connector 15 of FIG. 2a and the gripper end of a forceps 32. The forceps 32 holds the connector 15 and inserts the connector 15 in the direction of arrow B into the lumen 33 of graft vessel 30.

FIG. 3b shows that the pins 17 and 18 are pierced from inside the lumen of graft vessel 30 through the wall 34 of the graft vessel 30 to outside the graft vessel 30. The insertion movement in the direction B is continued to shift the connector further in the direction of insertion B up to the pins fully passed through the wall 34 of the graft vessel, see FIG. 3c.

In FIG. 3c, the pins have fully passed through the wall 34 of the graft vessel 30 and lie essentially parallel to the wall of the graft vessel 30. The ring 16 of the connector lies inside the graft vessel and is shown by dashed line for illustrative purpose.

FIG. 3d shows that, in the region surrounded by the ring 16, an opening 35 has been made through the wall 34 of the graft vessel 30. This opening serves to allow blood to enter or leave the graft vessel at this place in order to allow blood flowing into or from the recipient vessel. This opening 35 can be made with an Excimer laser as mentioned earlier several times. The opening can also be made with another tool like a knife.

FIG. 3d further shows that sutures 36 can be used to attach the ring 16 against the inner side of the wall 34 of the graft vessel 30. The sutures 36 can be provided before or after making the opening 35.

It is to be noted that the opening 35 does not need to be made at this stage, it is also conceivable to make the opening after attaching the assembly of graft vessel 30 and connector 15 to the recipient vessel.

FIG. 4 illustrates a second method of making an assembly according to the invention. The main difference between the method illustrated with FIG. 3 and the method illustrated with FIG. 4, is that in the method of FIG. 3 the ring of the connector is arranged inside the graft vessel and the pins 17, 18 of the connector are arranged outside the graft vessel, whilst in the method of FIG. 4 the ring and pins are both arranged outside the graft vessel.

FIG. 4a shows a graft vessel 30, the connector 15 of FIG. 2a and the gripper end of a forceps 32. The forceps 32 holds the connector 15 and places the connector 15 in the direction of arrow C against the graft vessel 30 so that the ring 16 lies between the wall of the graft vessel 30 and the pins 17, 18.

FIG. 4b shows as a next step that an opening 35 has been made in the region of the wall of the graft vessel which surrounded by the ring. This opening 35 will later allow blood flow between the graft vessel and recipient vessel. Further the wall tissue of the graft vessel 30 in said region surrounded by the ring 16 is not removed, at least not fully removed. Parts 37, 38, and 39 of the wall tissue in said region are used to cover the inner side of the ring 16. Doing so the internal surface 40 of the parts 37, 38, 39 of wall tissue will face inwardly. After completing the anastomotic connection, it will be this internal surface 40 which contacts blood flowing through the anastomotic connection. This is favourable as this internal surface is the natural surface for contacting blood. As shown sutures 36 are used on the one hand to attach the ring 16 to the graft vessel and on the other hand to keep the parts 37, 38 and 39 against the inner side of the ring. It is noted that in case the parts 37, 38, 39 have, in radial direction of the ring, sufficient length, they might be folded around the ring and inserted into the gap between the ring 16 and pins 17, 18.

FIG. 5 shows how an assembly according to the invention can be attached to a recipient vessel in order to accomplish the side-to-side anastomosis. Although the assembly shown in FIG. 5 is an assembly made according to FIG. 4, it is noted that an assembly made according to FIG. 3 can also be attached to the recipient vessel in the manner as illustrated in FIG. 5.

FIG. 5a shows the assembly according to the invention. The parts 41 and 42 of the graft vessel 30, which lie adjacent the connector 15, are folded towards the axial axis A of the ring 16, or said differently these parts 41 and 42 are folded towards each other. This provided the operator, who has to make the anastomotic connection, a better view onto the connector 15.

FIG. 5b shows in addition a recipient vessel 50 and two foreceps 32. One of the foreceps holds the connector 15 at its ring. The other foreceps grips a part 51 of the vessel wall 52 of the recipient vessel and pulls this part 51 away from the longitudinal axis 53 of the recipient vessel 50. Although this gripping and pulling of the part 51 is optional, it makes it easier to pierce the pins 17 and 18 into the wall of the recipient vessel.

In order to pierce the pins 17 and 18 from outside the recipient vessel 50 through the wall 52 of the recipient vessel into the lumen of the recipient vessel, the assembly of graft vessel 30 and connector 15 is shifted in the direction of arrow D. Once the pointed free ends of the pins passed from the outside through the vessel wall 52, the connector is shifted further in the direction of arrow D. At the end of this shifting movement, the free ends of the pins 17, 18 can be pierced again through the vessel wall, but now from the inside to the outside. This condition is shown in FIG. 5d.

FIG. 5d further shows that a laser catheter 60 has been inserted into the part 42 of the graft vessel 40 to burn away that part of the wall of the recipient vessel lying in the region surrounded by the ring 16. For details of a suitable laser catheter 60 reference can be made to the earlier mentioned U.S. Pat. No. 5,964,750. FIG. 4 of this U.S. Pat. No. 5,964,750 shows more detailed the manner of function of the laser catheter tip during application of laser light. A ring shaped cut is burnt and the vessel part lying inside this cut is suctioned against a grid to remove it. U.S. Pat. No. 5,964,750 is in this respect incorporated by reference. After the opening for connection of the lumens of the graft vessel and recipient vessel have been made, the laser catheter is withdrawn in direction E and the part 42 is provided with a clip to prevent leakage of blood. Part 42 can also be closed by suturing, burning or other means in order to prevent blood from flowing out this end. During use of the laser catheter 60 also the part 41 of the graft vessel might be temporarily closed, for example by a clip.

It is noted that in case the graft vessel has sufficient length, it is also possible to use one graft vessel for a double bypass, also called a jump. Referring to FIG. 1, it is for example conceivable to connect one end of the graft vessel at 10 to the aorta, the other end at 14 to the coronary artery 4 and to connect the graft vessel somewhere in between its ends at the location 11 with coronary artery 3.

The present invention can also be phrased as worded in the next following clauses:
1] Assembly comprising on the one hand a graft vessel and on the other hand an anastomosis connector;
wherein the connector comprises a ring and two pins;
each pin having a free end and a fixed end connected to the ring;
the pins extending, viewed from the fixed end towards the free end, next to and set apart from each other;
each pin having an overlap section where the pin, viewed in axial direction of the ring, overlaps the plane bounded by the outer contour of the ring;
wherein the pins are arranged outside the graft vessel;
wherein the axial axis of the ring extends transverse to the wall of the graft vessel;
wherein the connector is attached to the graft vessel; and
wherein the ring lies, with its full circumference, against the wall of the graft vessel.
2] Assembly according to clause 1, wherein the graft vessel is separated from a human or animal body.
3] Assembly according to one of the preceding clauses, wherein the wall of the graft vessel is provided with an opening surrounded by the ring.
4] Assembly according to clause 3, wherein the opening has been formed by laser cutting an opening into the wall of the graft vessel.
5] Assembly according to one of the preceding clauses, wherein the connector is attached to the graft vessel by sutures attaching the ring onto the wall of the graft vessel.
6] Assembly according to one of the preceding clauses, wherein the connector is attached to the graft vessel by adhesive adhering the ring onto the wall of the graft vessel.
7] Assembly according to one of the preceding clauses, wherein the connector is attached to the graft vessel by the pins piercing through the wall of the graft vessel.
8] Assembly according to one of the preceding clauses, wherein the pins extend in the length direction of the graft vessel.
9] Assembly according to one of the preceding clauses, wherein the ring lies against the outer surface of the wall of the graft vessel.
10] Assembly according to clause 9, wherein a part of the wall of the graft vessel lying inside the region surrounded by the ring is folded outwardly to cover the inner side of the ring.
11] Assembly according to one of the preceding clause 1-8, wherein the ring lies against the inner surface of the wall of the graft vessel and wherein the pins lie outside the graft vessel and extend parallel to the outer surface of the wall of the graft vessel.
12] Assembly according to one of the preceding clauses, especially according to clause 11, wherein the graft vessel has a first end and a second end; wherein the pins, viewed from the fixed ends towards the free ends, extend towards the second end; and wherein the distance from the connector to the first end of the graft vessel is smaller than the distance from the connector to the second end.
13] Assembly according to clause 11, wherein the distance from the connector to the first end is at least 0.5 cm, such 1.0 cm or more.
14] Assembly according to one of the preceding clauses, wherein a rod like application member is inserted through an end of the graft vessel into the lumen of the graft vessel.
15] Assembly according to one of the preceding clauses, wherein, in a first position of the pins with respect to the ring, at least part of the overlap sections extend parallel to the ring.
16] Assembly according to one of the preceding clauses, wherein the pins are rigidly connected to the ring.
17] Assembly according to one of the clauses 1-15, wherein the pins are with respect to the ring moveable from a first position to a second position in order to form between on the one hand the ring and on the other hand the pins an opened jaw; and in which the pins are moveable from the second position to the first position towards the ring in order to close the jaw.
18] Assembly according to clause 17, wherein in the second position the overlap sections of the pins are at an angle with respect to the plane defined by the ring in order to form a said jaw which, viewed from the fixed end towards the free end, widens, and wherein the pins are swivelable, with respect to the ring, from the second position to the first position.
19] Assembly according to clause 17 or 18, wherein the connector further comprises a tensioning member which, when the pins are in the second position, pretensions the pins towards the first position.
20] Assembly according to one of the preceding clauses, wherein the free ends of the pins are arranged at a distance from the overlap section, which distance is at least 50% of the radius of the ring, in particular at least 100% of the radius of the ring.
21] Assembly according to clause 20, wherein the sections of the pins between the overlap section and the free end of each pin, are essentially parallel to each other.
22] Method of making an assembly comprising on the one hand a graft vessel and on the other hand an anastomosis connector;
wherein the connector comprises a ring and two pins; each pin having a free end and a fixed end connected to the ring; the pins extending, viewed from the fixed end towards the free end, next to and set apart from each other; each pin having an overlap section where the pin, viewed in axial direction of the ring, overlaps the plane bounded by the outer contour of the ring;
wherein the method comprises the step of:
   arranging the ring to lie, with its full circumference against the wall of the graft vessel and with its axial axis transverse to the wall of the graft vessel; and
   attaching the connector to the graft vessel.
23] Method according to clause 22, wherein the graft is, during this method, separated from human or animal body.
24] Method according to one of clauses 22-23, further comprising the step of:
   making an opening through the wall of the graft vessel in the region surrounded by the ring.
25] Method according to clause 24, wherein the step of making said opening comprises burning with a laser, such as an Excimer laser, an opening through the wall of the graft vessel.
26] Method according to one of clauses 22-24, wherein the step of attaching the connector to the graft vessel comprises suturing the ring onto the wall of the graft vessel.
27] Method according to one of clauses 22-26, wherein the step of attaching the connector to the graft vessel comprises adhering the ring onto the wall of the graft vessel.
28] Method according to one of clauses 22-27, wherein the step of attaching the connector to the graft vessel comprises piercing the pins through the wall of the graft vessel.
29] Method according to one of clauses 22-28, wherein the step of attaching the connector to the graft vessel comprises:
   inserting the connector into the lumen of the graft vessel with the free ends of the pins pointing in the direction of insertion;
   piercing the free ends of the pins from the inside of the graft vessel through the wall of the graft vessel to outside the graft vessel and subsequently shifting the connector further in the direction of insertion up to the pins fully passed through the wall of the graft vessel and the ring lies against the inner surface of the wall of the graft vessel.
30] Method according to one of clauses 22-28, wherein, in the step of attaching the connector to the graft vessel, the ring is placed against the outside of the graft vessel.
31] Method according to clause 30, wherein the step of attaching the connector to the graft vessel further comprises covering the inside of the ring with a part of the wall of the graft vessel lying inside the region surrounded by the ring.
32] Method of making a side-to-side anastomosis, comprising the step of:
   attaching an assembly according to one of clauses 1-21 to a recipient vessel by piercing the free ends of the pins from the outside of the recipient vessel through the wall of the recipient vessel into the lumen of the recipient vessel and subsequently sliding the assembly further up up to the pins fully passed through the wall of the recipient vessel; and
   subsequently making a flow connection between the lumen of the graft vessel and the lumen of the recipient vessel.
33] Method according to clause 32, wherein the step of making said flow connection comprises burning with a laser, such as an Excimer laser, an opening through the wall of the recipient vessel and/or graft vessel.
34] Method according to clause 33, wherein the laser comprises a tubular bundle of densely packed laser fibres arranged to burn a ring and a suction gripper arranged inside the tubular bundle for gripping the part bounded by the ring burnt with the laser, and wherein the step of burning comprises burning a ring shaped passage through the wall of the recipient vessel and/or graft vessel and suctioning the part bounded by the ring burnt into the tubular bundle.
35] Method according to one of clauses 32-34, wherein, during the step of attaching, the assembly is being manipulated by means of a rod like application member inserted through one end of the graft vessel into the lumen of the graft vessel.
36] Method according to clause 35, wherein the insertion direction of the application member is the same as the direction into which the pins of the connector extend.
37] Method according to clause 35 or 36, wherein the rod extends in the direction of the axial axis of the ring and wherein the part of the graft vessel extending from adjacent the connector in the direction of the other end of the graft vessel is kept parallel to the application member.
38] Method according to one of clauses 32-37, wherein, at the location where the anastomosis is to be made, a part of the wall of the recipient vessel is gripped and lifted or pulled, transverse to the longitudinal axis of the recipient vessel, away from said longitudinal axis.
39] Method according to one of clauses 32-38, wherein, during the step of attaching, the parts of the graft vessel adjacent the connector are folded towards the axial axis of the ring, during the step of attaching.
40] Method according to one of clauses 32-39, further comprising a step of providing a said assembly according to one of clauses 1-21.
41] Method according to clause 35, wherein the step of providing said assembly comprises the method according to one of clauses 22-31

1. Assembly comprising an anastomosis connector for use with a graft vessel;
wherein the connector comprises a ring and two pins;
each pin having a free end and a fixed end connected to the ring;
the pins extending, viewed from the fixed end towards the free end, next to and set apart from each other;
each pin having an overlap section where the pin, viewed in axial direction of the ring, overlaps the plane bounded by the outer contour of the ring;
wherein the pins are arranged outside the graft vessel;
wherein the axial axis of the ring extends transverse to the wall of the graft vessel;
wherein the connector is attached to the graft vessel; and
wherein the ring lies, with its full circumference, against the wall of the graft vessel.

2. Assembly according to claim 1, wherein the graft vessel is separated from a human or animal body.

3. Assembly according to claim 1, wherein the wall of the graft vessel defines an opening surrounded by the ring.

4. Assembly according to claim 3, wherein the opening has been formed by laser cutting an opening into the wall of the graft vessel.

5. Assembly according to claim 1, wherein the connector is attached to the graft vessel by sutures attaching the ring onto the wall of the graft vessel.

6. Assembly according to claim 1, wherein the connector is attached to the graft vessel by adhesive adhering the ring onto the wall of the graft vessel.

7. Assembly according to claim 1, wherein the connector is attached to the graft vessel by the pins piercing through the wall of the graft vessel.

8. Assembly according to claim 1, wherein the pins extend in the length direction of the graft vessel.

9. Assembly according to claim 1, wherein the ring lies against the outer surface of the wall of the graft vessel.

10. Assembly according to claim 9, wherein a part of the wall of the graft vessel lying inside the region surrounded by the ring is folded outwardly to cover the inner side of the ring.

11. Assembly according to claim 1, wherein the ring lies against the inner surface of the wall of the graft vessel and wherein the pins lie outside the graft vessel and extend parallel to the outer surface of the wall of the graft vessel.

12. Assembly according to claim 11, wherein the graft vessel has a first end and a second end; wherein the pins, viewed from the fixed ends towards the free ends, extend towards the second end; and
wherein the distance from the connector to the first end of the graft vessel is smaller than the distance from the connector to the second end.

13. Assembly according to claim 11, wherein the distance from the connector to the first end is at least 0.5 cm, such 1.0 cm or more.

14. Assembly according to claim 1, wherein a rod like application member is inserted through an end of the graft vessel into the lumen of the graft vessel.

15. Assembly according to claim 1, wherein, in a first position of the pins with respect to the ring, at least part of the overlap sections extend parallel to the ring.

16. Assembly according to claim 1, wherein the pins are rigidly connected to the ring.

17. Assembly according to claim 1, wherein the pins are with respect to the ring moveable from a first position to a second position in order to form between on the one hand the ring and on the other hand the pins an opened jaw; and in which the pins are moveable from the second position to the first position towards the ring in order to close the jaw.

18. Assembly according to claim 17, wherein in the second position the overlap sections of the pins are at an angle with respect to the plane defined by the ring in order to form a said jaw which, viewed from the fixed end towards the free end, widens, and wherein the pins are swivelable, with respect to the ring, from the second position to the first position.

19. Assembly according to claim 17, wherein the connector further comprises a tensioning member which, when the pins are in the second position, pretensions the pins towards the first position.

20. Assembly according to claim 1, wherein the free ends of the pins are arranged at a distance from the overlap section, which distance is at least 50% of the radius of the ring, in particular at least 100% of the radius of the ring.

21. Assembly according to claim 20, wherein the sections of the pins between the overlap section and the free end of each pin, are essentially parallel to each other

22. Method of making an assembly comprising an anastomosis connector for use with a graft vessel;
wherein the connector comprises a ring and two pins; each pin having a free end and a fixed end connected to the ring; the pins extending, viewed from the fixed end towards the free end, next to and set apart from each other; each pin having an overlap section where the pin, viewed in axial direction of the ring, overlaps the plane bounded by the outer contour of the ring;
wherein the method comprises the step of:
arranging the ring to lie, with its full circumference against the wall of the graft vessel and with its axial axis transverse to the wall of the graft vessel; and
attaching the connector to the graft vessel.

23. Method according to claim 22, wherein the graft is separated from human or animal body.

24. Method according to one of claim 22, further comprising the step of:
making an opening through the wall of the graft vessel in the region surrounded by the ring.

25. Method according to claim 24, wherein the step of making said opening comprises burning with a laser, such as an Excimer laser, an opening through the wall of the graft vessel.

26. Method according to claim 22, wherein the step of attaching the connector to the graft vessel comprises suturing the ring onto the wall of the graft vessel.

27. Method according to one of claim 22, wherein the step of attaching the connector to the graft vessel comprises adhering the ring onto the wall of the graft vessel.

28. Method according to one of claim 22, wherein the step of attaching the connector to the graft vessel comprises piercing the pins through the wall of the graft vessel.

29. Method according to claim 22, wherein the step of attaching the connector to the graft vessel comprises:
inserting the connector into the lumen of the graft vessel with the free ends of the pins pointing in the direction of insertion;
piercing the free ends of the pins from the inside of the graft vessel through the wall of the graft vessel to outside the graft vessel and subsequently shifting the connector further in the direction of insertion up to the pins fully passed through the wall of the graft vessel and the ring lies against the inner surface of the wall of the graft vessel.

30. Method according to claim 22, wherein, in the step of attaching the connector to the graft vessel, the ring is placed against the outside of the graft vessel.

31. Method according to claim 30, wherein the step of attaching the connector to the graft vessel further comprises covering the inside of the ring with a part of the wall of the graft vessel lying inside the region surrounded by the ring.

32. Method of making a side-to-side anastomosis, comprising the step of:
attaching an assembly according to claim 1 to a recipient vessel by piercing the free ends of the pins from the outside of the recipient vessel through the wall of the recipient vessel into the lumen of the recipient vessel and subsequently sliding the assembly further up up to the pins fully passed through the wall of the recipient vessel; and
subsequently making a flow connection between the lumen of the graft vessel and the lumen of the recipient vessel.

33. Method according to claim 32, wherein the step of making said flow connection comprises burning with a laser, such as an Excimer laser, an opening through the wall of the recipient vessel and/or graft vessel.

34. Method according to claim 33, wherein the laser comprises a tubular bundle of densely packed laser fibres arranged to burn a ring and a suction gripper arranged inside the tubular bundle for gripping the part bounded by the ring burnt with the laser, and wherein the step of burning comprises burning a ring shaped passage through the wall of the recipient vessel and/or graft vessel and suctioning the part bounded by the ring burnt into the tubular bundle.

35. Method according to claim 32, wherein, during the step of attaching, the assembly is being manipulated by means of a rod like application member inserted through one end of the graft vessel into the lumen of the graft vessel.

36. Method according to claim 35, wherein the insertion direction of the application member is the same as the direction into which the pins of the connector extend.

37. Method according to claim 35, wherein the rod extends in the direction of the axial axis of the ring and wherein the part of the graft vessel extending from adjacent the connector in the direction of the other end of the graft vessel is kept parallel to the application member.

38. Method according to claim 32, wherein, at the location where the anastomosis is to be made, a part of the wall of the recipient vessel is gripped and lifted or pulled, transverse to the longitudinal axis of the recipient vessel, away from said longitudinal axis.

39. Method according to claim 32, wherein, during the step of attaching, the parts of the graft vessel adjacent the connector are folded towards the axial axis of the ring, during the step of attaching.

40. Method according to claim 32, further comprising a step of providing a said assembly according to claim 1.

41. Method according to claim 35, wherein the step of providing said assembly comprises the method according to claim 22.
